(19) United States
(12) Patent Application Publication
(10) Pub. No.: US 2011/0046618 A1
(43) Pub. Date: Feb. 24, 2011

(54) METHODS AND SYSTEMS FOR TREATING OCCLUDED BLOOD VESSELS AND OTHER BODY CANNULA

(76) Inventors: Christopher D. Minar, New Prague, MN (US); Pete Skujins, Minneapolis, MN (US); Alan D. Eskuri, Hanover, MN (US); Hong Cao, Savage, MN (US); Khamla Vongphakdy, Big Lake, MN (US)

Correspondence Address: HARNESS, DICKEY, & PIERCE, P.L.C, 7700 Bonhomme, Suite 400, ST. LOUIS, MO, 63105, US

(21) Appl. No.: 12/847,062

(22) Filed: Jul. 30, 2010

Related U.S. Application Data
(60) Provisional application No. 61/231,055, filed on Aug. 4, 2009.

(51) Int. Cl. A61B 18/00

(52) U.S. Cl. 606/41

(57) ABSTRACT

A method of treating an occluded blood vessel includes sensing the impedance at a plurality of locations around the circumference of the blood vessel at least two different frequencies to identify vascular occlusive material, and distinguish vascular occlusive material from the vessel wall.

### FIELD

The present disclosure relates to methods and systems for treating occluded blood vessels.

### BACKGROUND

This section provides background information related to the present disclosure which is not necessarily prior art.

Occluded blood vessels, particularly the blood vessels of the heart, is a common medical condition. Methods and systems for opening these vessels using mechanical cutting instruments, RF ablation, or laser ablation to remove the vascular occlusive material have been developed to improve blood flow. While effective, a problem with most of these methods and systems is the risk of perforation of the blood vessel wall.

Methods and systems, such as OCR (optical coherence reflectometry) have been developed for distinguishing vascular occlusive material from the vessel wall to improve the efficiency of removing vascular occlusive material, and reduce inadvertent damage to the vessel wall. However these methods and systems do not always reliably distinguish between vascular occlusive material and the vessel walls, and the available methods and systems for removing vascular occlusive material are not always been reliably to remove only vascular occlusive material.

The use of RF ablation to vaporize vascular occlusive materials is described in Bales, U.S. Pat. No. 4,682,596, and Rosar, U.S. Pat. No. 5,300,068, incorporated herein by reference.

Generally, the methods and systems of the invention are useful in treating occluded blood vessels.

In one embodiment, a method of treating an occluded blood vessel is provided that comprises sensing the impedance at a plurality of locations around the circumference of the blood vessel at least two different frequencies to identify vascular occlusive material, and distinguish vascular occlusive material from the vessel wall.

Once the vascular occlusive material has been identified, ablative energy can be applied only to the identified vascular occlusive material to remove it with reduced risk of damaging the blood vessel walls. This can be done, for example, by moving an electrode toward a location where vascular occlusive material has been identified, and using the electrode to apply ablative energy to the vascular occlusive material at the location.

The impedance sensing at least two frequencies can be done simultaneously or contemporaneously in quick succession. For example, in the case of two frequencies, the sensing can be done within 50 ms, and preferably within 20 ms, and more preferably within 10 ms. The impedance can be sensed in a mutlipolar mode between two electrodes disposed in the blood vessel, which can be disposed on the same medical device, or two separate medical devices, or in a unipolar mode with an electrode in the blood vessel and an electrode outside of the blood vessel.

In another embodiment, a method of treating an occluded blood vessel is provided that comprises positioning a medical device in the blood vessel. The medical device having a plurality of electrodes around its circumference, which can be used to sense the impedance at a plurality of locations around the circumference of the blood vessel with the plurality of electrodes at least two different frequencies to identify vascular occlusive material, and distinguish vascular occlusive material from the vessel wall.

Once the vascular occlusive material has been identified, ablative energy can be applied only to the identified vascular occlusive material to remove it with reduced risk of damaging the blood vessel walls. This can be done, for example, by moving an electrode toward a location where vascular occlusive material has been identified, and using the electrode to apply ablative energy to the vascular occlusive material at the location.

The impedance sensing at least two frequencies can be done simultaneously, or contemporaneously in quick succession. For example, in the case of two frequencies, the sensing can be done within 50 ms, and preferably within 20 ms, and more preferably within 10 ms. The impedance can be sensed in a mutlipolar modem between two electrodes disposed in the blood vessel, which can be disposed on the same medical device, or two separate medical devices, or in a unipolar mode with an electrode in the blood vessel and an electrode outside of the blood vessel.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a side elevation view of a catheter in accordance with a first preferred embodiment of the invention;

FIG. 2 is a distal end elevation view of the catheter;

FIG. 3 is a side elevation view of the catheter, showing a electrophysiology guide wire extending from its distal end;

FIG. 4 is a side elevation view of a guide wire in accordance with a second preferred embodiment;

FIG. 5 is a side elevation of an alternate construction of the guide wire of the second embodiment;

FIG. 6 is a distal end elevation view of the guide wire of FIG. 5;

FIG. 7 is a schematic diagram showing the impedance measuring schemes with the catheters and guide wires of some of the embodiments of the invention;

FIG. 8 is a perspective view of the distal end of a catheter in accordance with a third preferred embodiment of the invention;

FIG. 9 is a side elevation view of an alternate construction of the catheter of the third embodiment;

FIG. 10 is a distal end elevation view of the catheter of FIG. 9; and

FIG. 11 is a side elevation view of a catheter constructed according to the principles of a fourth preferred embodiment.

Corresponding reference numerals indicate corresponding parts throughout the several views of the drawings.

### DETAILED DESCRIPTION

Example embodiments will now be described more fully with reference to the accompanying drawings.

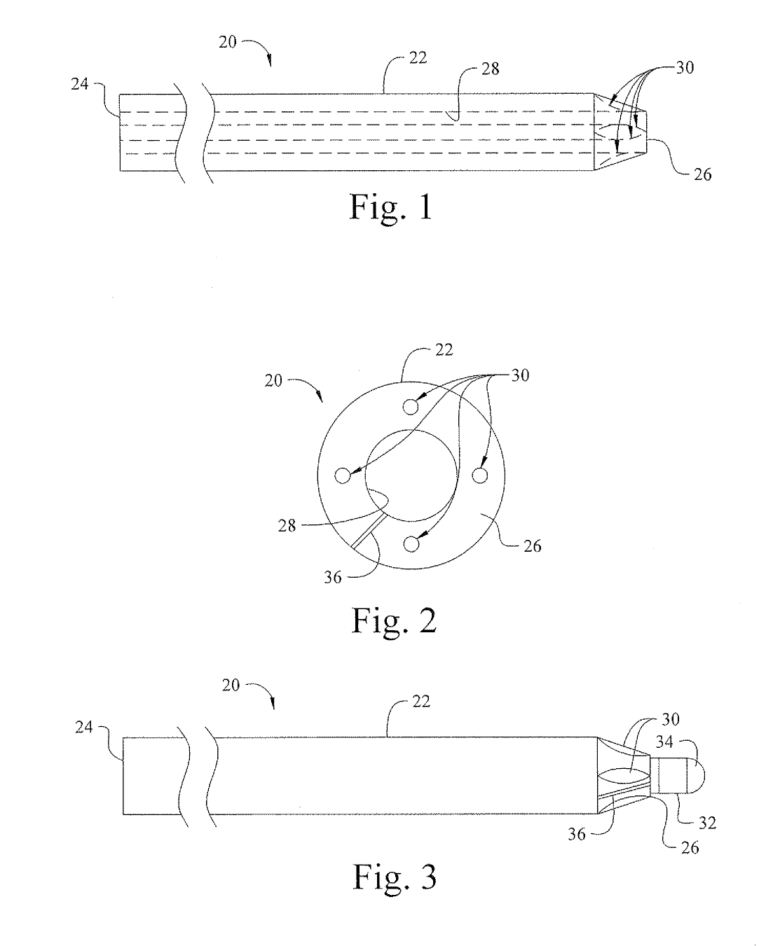

A first preferred embodiment of a system for removing vascular occlusive material is indicated generally as 20 in FIGS. 1-3. The system 20 comprises a catheter 22 having a proximal end 24 and a distal end 26, and a lumen 28 there between. A plurality (in this preferred embodiment at least four) electrodes 30 are disposed on the catheter 22, adjacent the distal end 26. The electrodes 30 are preferably equally spaced around the circumference of the catheter 22.

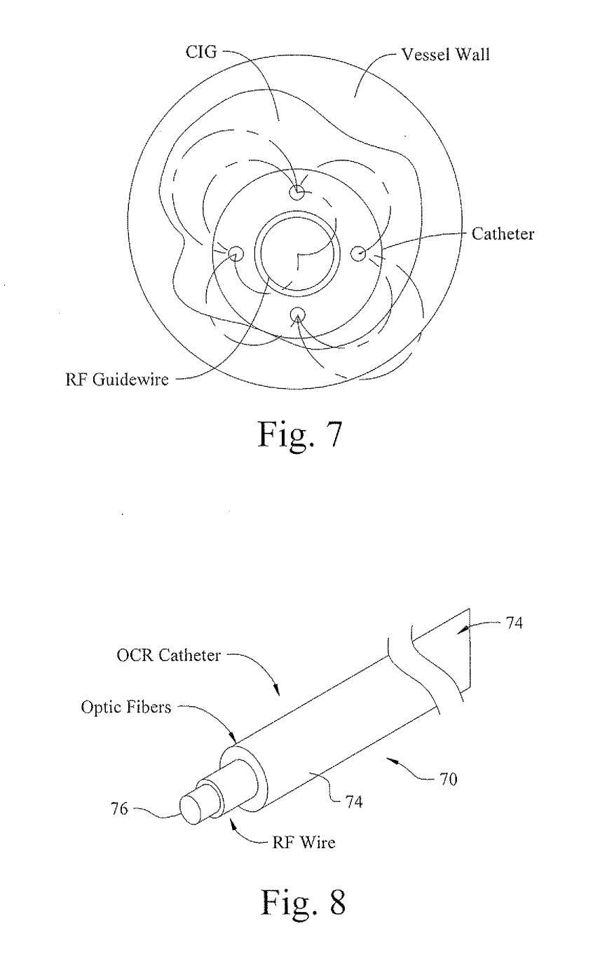

An ablation RF guide wire 32 (FIG. 3), having an electrode 34 on its distal end, can be advanced through the lumen 28. As shown in FIG. 7, when the catheter 22 and guide wire 32 are deployed in an occluded vessel, the impedance of the tissue surrounding the catheter and guide wire can be measured between the electrode 34 on the guide wire 32, and each of the electrodes 30 on the catheter 22. The impedance of the tissue surrounding the catheter 22 can alternatively or additionally be measured between the various electrodes 30 on the catheter 22. The impedance of the tissue surrounding the catheter 22 can alternatively or additionally be measured between each of the electrodes 30 on the catheter 22 and another electrode disposed in the body or on the surface of the body.

In accordance with the principles of this invention, the impedance is measured at least two frequencies, for example 10 kHz and 100 kHz, however, the impedance could be measured at different or additional frequencies. The inventors have observed that the impedance of living tissue generally decreases as measurement frequency increases, but that the impedance of fat or calcification, typical of vascular occlusive material remains relatively stable as the measurement frequency increases. Thus, while a single impedance measurement can be used to distinguish between vascular occlusive material and the blood vessel wall, measuring the impedance at multiple frequencies improves the discrimination between occlusive material and the vessel wall.

The system 20 preferably also includes a switching unit (not shown), that switches to select the electrode (in unipolar measurements) or electrodes (in bipolar measurements) used to measure the impedance. After impedance measurement or measurements, the switching unit switches to a different electrode or electrode pair. The system 20 also includes a multiple impedance measuring circuit for measuring the impedance at two or more frequencies. The impedance can be measured at multiple frequencies simultaneously, and the measurements obtained by filtering, or the impedance measured at a succession of frequencies contemporaneously (i.e. sufficiently close in time that the measurements correspond to the same location). For example, a single impedance measurement could be made in 5 ms at each frequency. The making two measurements (one at each of two frequencies) per location would allow one hundred locations to be measured per second. This rate is sufficiently fast that movement (e.g. of the heart or of the subject) will still not prevent measurements at two frequencies at each location. In fact, the rate could be substantially slower, or measurements at additional frequencies could be made.

In the presence of vascular occlusive material, the impedance measurements should indicate that at least one of the electrodes 30 is oriented more toward vascular occlusive material (i.e., having a higher impedance).

An orientation system, for example a radiopaque marker 36 on the catheter 22 permits the user to discern the orientation of the electrode with respect to the imaging and navigation systems so that the user can navigate the guide wire 32 toward the vascular occlusive material (and away from the healthy vessel wall). The electrode 34 on the guide wire 32 can then be used to ablate the vascular occlusive material.

Alternatively, the electrodes 30 on the catheter 22 can be used to ablate the vascular occlusive material, eliminating the need to properly position the guide wire 32. The electrode or electrodes 30 adjacent to the vascular occlusive material ablate the adjacent vascular occlusive material, and the catheter 22 can be navigated into the volume created by the removal of the vascular occlusive material, and the process repeated.

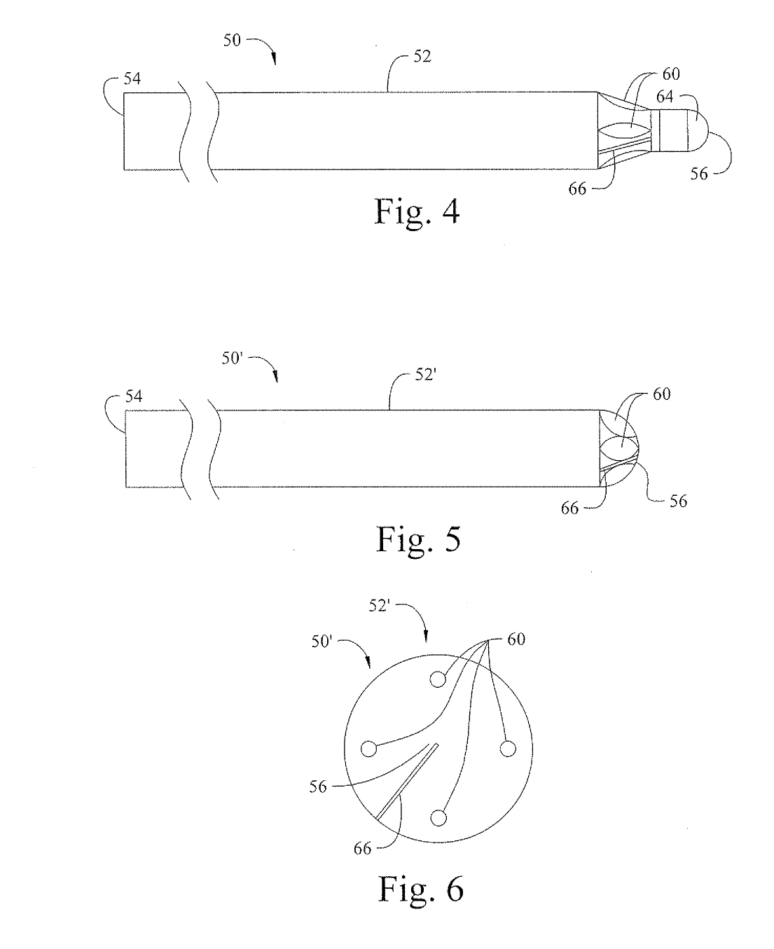

A second preferred embodiment of a system for removing vascular occlusive material is indicated generally as 50 in FIG. 4, or 50′ in FIGS. 5-6. The system 50 (or 50′) comprises a guide wire 52 or 52′ having a proximal end 54 and a distal end 56. A plurality (in this preferred embodiment at least four) electrodes 60 are disposed on the guide wire 52 or 52′, adjacent the distal end 56. The electrodes 30 are preferably equally spaced around the circumference of the guide wire 52 or 52′.

As shown in FIG. 4, an ablation electrode 64 can be positioned on the distal end 56 of the guide wire 52. The impedance of the tissue surrounding the guide wire 52 can be measured between the electrode 64 on the distal end 56 of the guide wire 52, and each of the electrodes 60 adjacent the distal end of the guide wire. The impedance of the tissue surrounding the guide wire 52 can alternatively or additionally be measured between the various electrodes 60 on the guide wire 52. The impedance of the tissue surrounding the guide wire 52 can alternatively or additionally be measured between each of the electrodes 60 on the guide wire 52 and another electrode disposed in the body or on the surface of the body.

As shown in FIGS. 5 and 6, the guide wire 52 does not have the extension or ablation electrode 64. The impedance of the tissue surrounding the guide wire 52′ can alternatively or additionally be measured between the various electrodes 60 on the guide wire 52′. The impedance of the tissue surrounding the guide wire 52′ can alternatively or additionally be measured between each of the electrodes 60 on the guide wire 52′ and another electrode disposed in the body or on the surface of the body.

As discussed above, in accordance with the principles of this invention, the impedance is measured at least two frequencies.

The system 50 preferably also includes a switching unit (not show) that switches to select the electrode (in unipolar measurements) or electrodes (in bipolar measurements). After impedance measurement or measurements, the switching unit switches to a different electrode or electrode pair. The system 50 also includes a multiple impedance measuring circuit for measuring the impedance at two or more frequencies. The impedance can be measured at multiple frequencies simultaneously, and the measurements obtained by filtering, or the impedance measured at a succession of frequencies contemporaneously (i.e. sufficiently close in time that the measurements correspond to the same location).

In the presence of vascular occlusive material, the impedance measurements should indicate that at least one of the electrodes 60 is oriented more toward vascular occlusive material (i.e., having a higher impedance).

An orientation system, for example a radiopaque marker 66 on the guide wire 52 permits the user to discern the orientation of the electrode with respect to the imaging and navigation systems so that the user can navigate the guide wire 52 toward the vascular occlusive material (and away from the healthy vessel wall). The electrode 64 on the guide wire 52 can then be used to ablate the vascular occlusive material.

The electrode 64 on guide wire 52 can be used to ablate the vascular occlusive material, eliminating the need to properly position the distal end 56 of the guide wire 52. Alternatively or additionally, the electrodes 60 on the guide wire 52 or 52′ adjacent to the vascular occlusive material ablate the adjacent vascular occlusive material. Then the guide wire 52 or 52′ can be navigated into the volume created by the removal of the vascular occlusive material, and the process repeated.

A third preferred embodiment of a system for removing vascular occlusive material is indicated generally as 70 in FIGS. 8-10. The system 70 comprises a catheter 72 having a proximal end 74 and a distal end 76, and a lumen 78 there between. A plurality (in this preferred embodiment at least four) fiber optic elements 80 are disposed on the catheter 72, adjacent the distal end 76. The elements 80 are preferably equally spaced around the circumference of the catheter 72. The fiber optic elements can be used to detect vascular occlusive material with OCR.

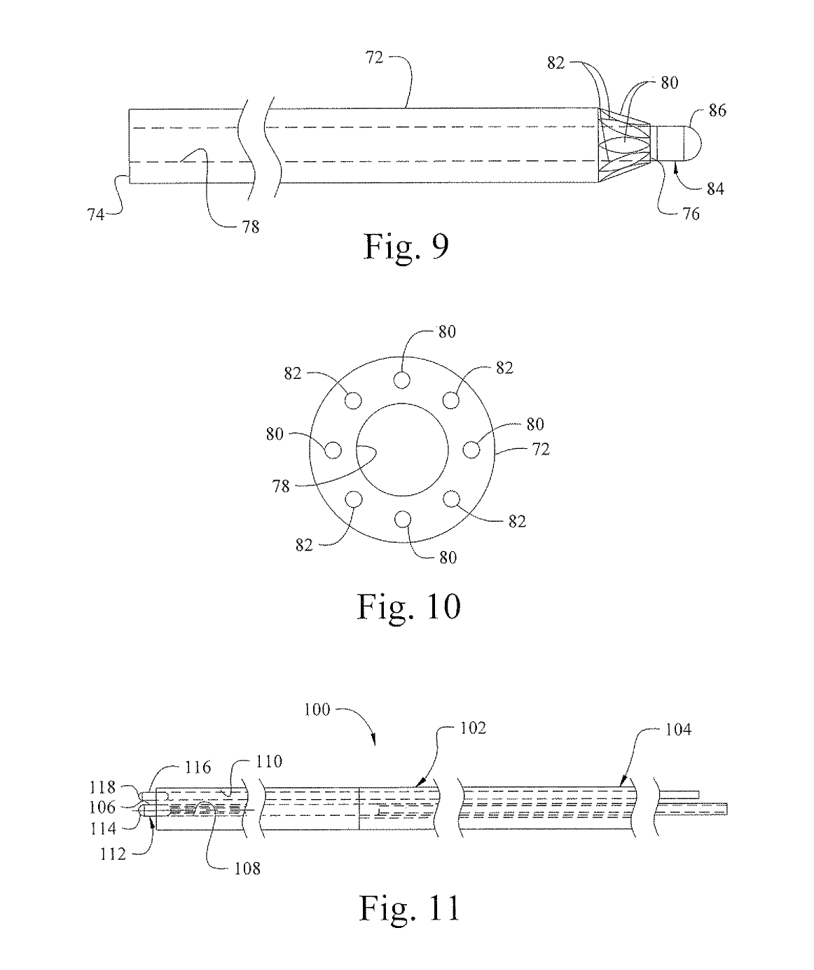

Preferably, as shown in FIGS. 9 and 10, in addition to the fiber optic elements 80, a plurality (in this preferred embodiment at least four) electrodes 82 are disposed on the catheter 72, adjacent the distal end 76. The electrodes 82 are preferably equally spaced around the circumference of the catheter 72. An ablation RF guide wire 84, having an electrode 86 on its distal end, can be advanced through the lumen 78.

The fiber optic elements 80 alone, or the combination of the fiber optic elements and the electrodes 82, can be used to detect vascular occlusive material. The fiber optic elements 80 can be used to detect vascular occlusive material through optical coherence reflectometry. The electrodes 82 can be used to detect vascular occlusive material by measuring impedance. The impedance of the tissue surrounding the catheter 72 and guide wire 84 can be measured between the electrode 86 on the guide wire 84, and each of the electrodes 82 on the catheter 72. The impedance of the tissue surrounding the catheter 72 can alternatively or additionally be measured between the various electrodes 82 on the catheter 72. The impedance of the tissue surrounding the catheter 72 can alternatively or additionally be measured between each of the electrodes 82 on the catheter 72 and another electrode disposed in the body or on the surface of the body.

In accordance with the principles of this invention, the impedance is measured at least two frequencies.

The system 20 preferably also includes a switching unit (not show) that switches to select the electrode (in unipolar measurements) or electrodes (in bipolar measurements). After impedance measurement or measurements, the switching unit switches to a different electrode or electrode pair. The system 20 also includes a multiple impedance measuring circuit for measuring the impedance at two or more frequencies. The impedance can be measured at multiple frequencies simultaneously, and the measurements obtained by filtering, or the impedance measured at a succession of frequencies contemporaneously (La, sufficiently close in time that the measurements correspond to the same location).

In the presence of vascular occlusive material, the impedance measurements should indicate that at least one of the electrodes 30 is oriented more toward vascular occlusive material (i.e., having a higher impedance).

An orientation system, for example a radiopaque marker 88 on the catheter 72, permits the user to discern the orientation of the electrode with respect to the imaging and navigation systems so that the user can navigate the guide wire 84 toward the vascular occlusive material (and away from the healthy vessel wall). The electrode 86 on the guide wire 84 can then be used to ablate the vascular occlusive material.

Alternatively, the electrodes 82 on the catheter 72 can be used to ablate the vascular occlusive material, eliminating the need to properly position the guide wire 84. The electrode or electrodes 82 adjacent to the vascular occlusive material ablate the adjacent vascular occlusive material, and the catheter 72 can be navigated into the volume created by the removal of the vascular occlusive material, and the process repeated. The combination of both OCR and impedance measurement can provide a superior detection of vascular occlusive material, and discrimination between such material and the healthy wall of the vessel.

A fourth preferred embodiment of a system for removing vascular occlusive material is indicated generally as 100 in FIG. 11. The system 100 comprises a catheter 102 having a proximal end and a distal end 76, and at least one, but preferably two lumens 108 and 110 there between. The lumen 108 preferably extends along the center axis of the catheter 102, while the lumen 110 extends parallel to the central axis, but is substantially offset therefrom. (Alternatively both lumens 108 and 110 can be parallel to, but offset from, the axis).

An ablation guide wire 112 having an electrode 114 on its distal end, can be disposed in lumen 108. An electrode wire 116 having an electrode 118 on its distal end, can be disposed in lumen 110. The catheter 102 is adapted to be rotated and advanced, or more preferably rotated and retracted through a subject's vasculature. The electrodes 114 and 118 can be used to measure the impedance of the adjacent tissue, which in accordance with the principles of this invention can be done at least two frequencies. The impedance can be measured between the electrodes 114 and 118, or between one of the electrodes 114 and 118 and another electrode disposed in or on the surface of the subject. Preferably, the electrode 118, which is off-center, is used in the impedance measurement. The axial and rotational movement of the catheter 102 allows vascular occlusive material to be identified around the circumference of the vessel, and along its length. The speed of the axial movement and of the rotation can be coordinated with the impedance sensing so that at least two impedance measurements, each at a different frequency, are conducted at each location.

As vascular occlusive material is identified, the electrode 114 can be used to ablate it. Alternatively or additionally, the electrode 118 could be used for ablation as well. Rather than ablate the material as it is identified, a section of the blood vessel can be characterized, and the catheter 102, or some other device, inserted back into the characterized section to remove the vascular occlusive material that was identified.

While this description relates primarily to blood vessels and devices and methods can be used in other body lumens, including biliary, renal, pulmonary, and fallopian ducts.

The foregoing description of the embodiments has been provided for purposes of illustration and description. It is not intended to be exhaustive or to limit the invention. Individual elements or features of a particular embodiment are generally not limited to that particular embodiment but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

Example embodiments are provided so that this disclosure will be thorough, and will fully convey the scope to those who are skilled in the art. Numerous specific details are set forth, such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a”, “an”, and “the”, may be intended to include the plural forms as well, unless the context clearly indicates otherwise. The terms “comprises”, “comprising”, “including”, and “having”, are inclusive and therefore, specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on” “engaged to”, “connected to”, or “coupled to” another element or layer, it may be directly on, engaged, connected, or coupled to the other element or layer, or intervening elements or layers may be present. In contrast, when an element is referred to as being “directly on”, “directly engaged to”, “directly connected to”, or “directly coupled to” another element or layer, there may be no intervening elements or layers present. Other words used to describe the relationship between elements should be interpreted in a like fashion (e.g., “between” versus “directly between”, “adjacent” versus “directly adjacent”, etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer, or section from another region, layer, or section. Terms such as “first”, “second”, and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner”, “outer”, “beneath”, “below”, “lower”, “above”, “upper”, and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

What is claimed is:

1. A method of treating an occluded blood vessel, the method comprising:
sensing the impedance at a plurality of locations around the circumference of the blood vessel at least two different frequencies to identify vascular occlusive material, and distinguish vascular occlusive material from the vessel wall.

2. The method of claim 1 further comprising applying ablative energy to the identified vascular occlusive material.

3. The method of claim 2 further comprising moving an electrode toward a location where vascular occlusive material has been identified, and applying ablative energy to the vascular occlusive material at the location.

4. The method of claim 1 wherein sensing the impedance at least two frequencies is done simultaneously.

5. The method of claim 1 wherein sensing the impedance at least two frequencies is done contemporaneously.

6. The method of claim 1 wherein sensing the impedance at least two frequencies is done within 10 ms.

7. The method of claim 1 wherein the impedance is sensed between two electrodes disposed in the blood vessel.

8. The method of claim 1 wherein the impedance is sensed between an electrode in the blood vessel and an electrode outside of the blood vessel.

9. The method of claim 1 wherein the two frequencies are sufficiently different to yield different impedance measurements for the living tissue of the vessel.

10. The method of claim 9 wherein the two frequencies include about 10 kHz and 100 kHz.

11. A method of treating an occluded blood vessel, the method comprising:
positioning a medical device in the blood vessel, the medical device having a plurality of electrodes around its circumference; and
sensing the impedance at a plurality of locations around the circumference of the blood vessel with the plurality of electrodes at least two different frequencies to identify vascular occlusive material, and distinguish vascular occlusive material from the vessel wall.

12. The method of claim 11 further comprising applying ablative energy to the identified vascular occlusive material.

13. The method of claim 11 further comprising using at least one of the electrodes on the medical device to apply ablative energy to the identified vascular occlusive material.

14. The method of claim 11 wherein sensing the impedance at least two frequencies is done simultaneously.

15. The method of claim 11 wherein sensing the impedance at least two frequencies is done contemporaneously.

16. The method of claim 11 wherein sensing the impedance at least two frequencies is done within 10 ms.

17. The method of claim 11 wherein the impedance is sensed between two electrodes disposed in the blood vessel.

18. The method of claim 17 wherein the two electrodes are disposed on the same medical device.

19. The method of claim 17 wherein the two electrodes are disposed on different medical devices.

20. The method of claim 11 wherein the impedance is sensed between an electrode in the blood vessel and an electrode outside of the blood vessel.

21. The method of claim 11 wherein the two frequencies are sufficiently different to yield different impedance measurements for the living tissue of the vessel.

22. The method of claim 11 wherein the two frequencies include about 10 kHz and 100 kHz.

23. A method of identifying vascular occlusive material in a vessel, comprising rotating and axially moving a electrode device through the vessel and measuring the impedance at points along the length of the device at least two frequencies, to distinguish between vascular occlusive material and vessel wall.

24. The method of claim 23 further comprising making a three dimensional map of the location of vascular occlusive material based upon the impedance measurements.
